(19) United States
(12) Patent Application Publication
(10) Pub. No.: US 2025/0064536 A1
(43) Pub. Date: Feb. 27, 2025

(54) SYSTEM AND METHOD FOR DOCKING CONNECTOR FOR ROBOTIC SURGICAL SYSTEM

(71) Applicant: Jerome Canady Research Institute for Advanced Biological and Technological Sciences, Takoma Park, MD (US)

(72) Inventors: Jerome Canady, Lakeland, FL (US); Changhan Jun, Hyattsville, MD (US); Taisen Zhuang, Rockville, MD (US)

(21) Appl. No.: 18/815,641

(22) Filed: Aug. 26, 2024

Related U.S. Application Data
(60) Provisional application No. 63/534,759, filed on Aug. 25, 2023.

(51) Int. Cl. A61B 34/30; A61B 17/00; B25J 15/04

(52) U.S. Cl. CPC A61B 34/30; B25J 15/0408; A61B 2017/00203; A61B 2017/00477; A61B 2034/301

(57) ABSTRACT

A robotic medical surgical system docking connector. The docking connector has a housing, a plurality of controllers in the housing, a plurality of electrical interfaces in the housing, an interface for connecting the housing to a robotic surgical arm; and a mechanical interface for connecting the housing to a robotic surgical end effector. The housing has first and second handles, a programmable push button on at least one of the first and second handles; and a plurality of cable connectors. The plurality of electrical interfaces may comprise, a digital input output interface, an analog input output interface, a CANopen interface, a USB (5V,GND, Sig1,2) interface; and a programmable MCU interface.

### CROSS-REFERENCE TO RELATED APPLICATIONS

The present application claims the benefit of the filing date of U.S. Provisional Patent Application Ser. No. 63/534,759 filed by the present inventors on Aug. 25, 2023.

The aforementioned provisional patent application is hereby incorporated by reference in its entirety.

### STATEMENT REGARDING FEDERALLY SPONSORED RESEARCH OR DEVELOPMENT

None.

### BACKGROUND OF THE INVENTION

### Field of the Invention

The present invention relates to robotic surgical systems, and more specifically, a robotic surgical navigation system.

### Brief Description of the Related Art

Within the scope of minimally-invasive surgery, such as endoscopic or laparoscopic surgery, access to the operating site is made via small incisions in the body of the patient (such as the abdomen or thorax), in which the practitioner places a cannula formed by a tube whereof the diameter typically varies from 3 to 15 mm, through which the practitioner can insert into the body of the patient either an endoscope for obtaining a video image on a monitor, or long and fine instruments for performing a procedure at the operating site.

Manipulation of an intervention device through small incisions or through a natural orifice requires in both cases to move it around a fixed point or center of motion, which corresponds to the incision or natural orifice itself. Such incision or natural orifice is herein generally referred to as a point of penetration in the patient.

Since the surgeon generally has both hands occupied by the surgical instruments, an assistant is necessary to maintain any other intervention device in a desired position, in particular the endoscope that is used to guide the surgeon in his surgery.

Robotic systems have been developed to handle and displace the endoscope in the place of the assistant. Examples of modern systems include those disclosed in U.S. Pat. No. 10,639,066 entitled “System for Controlling Displacement of an Intervention Device,” which discloses a system for controlling displacement of an intervention device having an end for inserting in a patient body, including a base in a fixed position relative to the patient. A first portion has an arc member and is pivotally mounted on the base around a first axis (A1). A second portion includes a support member and a carrier member. The support member partially rotates around a second axis (A2). A third portion includes a holding member, and a sliding member mounted on the support member along a translation axis (A.sub.T). The holding member is arranged so that translation of the sliding member causes the intervention device to translate along a third axis (A3). The third axis (A3) is parallel to and offset from the translation axis (A.sub.T). When the carrier member is positioned halfway of the arc member, the first (A1), second (A2) and third (A3) axes are orthogonal.

WO2022/187639 discloses a system and method for controlling a dosage of cold plasma generated multi-species delivered to a patient. The distance of the CAP probe should be kept constant about 1.5-2.5 mm as well as the treating time and treating area should be controlled during the procedure. A robotic system such as is disclosed in U.S. Pat. No. 10,639,066 will have a quick attachable connection to the CAP probe, and the robotic system will keep a constant distance from CAP probe tip's end to target tissue and at same time provide a surface scan with computer planned controllable surface treatment area, treating time and step distance between two return scans.

Serial robot arms with 5 DoF-7 DoF are often used to position specially designed end-effectors for surgical applications. For example, the KUKA LBR Med robot is a 7-axis lightweight mobile medical robot. Surgical end-effectors include minimally invasive instruments, and the end-effectors are usually designed to pose less than 3 DoF such as Viky system that was developed to manipulate endoscopic/laparoscopic surgical instrument, but also can be applicable for open surgeries. The electro-mechanical end-effectors are controlled by their own motion controllers and console/operating systems, and it is required to configure separate cable connections along with the serial robot arm.

### SUMMARY OF THE INVENTION

In a prefer embodiment, the present invention is a robotic medical surgical system docking connector. The docking connector has a housing, a plurality of controllers in the housing, a plurality of electrical interfaces in the housing, an interface for connecting the housing to a robotic surgical arm; and a mechanical interface for connecting the housing to a robotic surgical end effector. The housing has first and second handles, a programmable push button on at least one of the first and second handles; and a plurality of cable connectors. The plurality of electrical interfaces may comprise, a digital input output interface, an analog input output interface, a CANopen interface, a USB (5V,GND, Sig1,2) interface; and a programmable MCU interface.

In another preferred embodiment, the present invention is a robotic medical surgical system having a robotic surgical arm, a robotic surgical end effector and a docking connector configured to connect the robotic surgical arm to the robotic surgical end effector. The docking connector has a docking connector has a housing, an interface connecting the housing to the robotic surgical arm; and a mechanical interface for connecting the housing to a robotic surgical end effector. The housing has first and second handles; a programmable push button on at least on of the first and second handles, and a plurality of cable connectors. The docking connector further has a plurality of controllers in the housing; and a plurality of electrical interfaces in the housing. A plurality of electrical cables connect the robotic surgical end effector to the electrical interfaces in the docking connector.

The plurality of electrical interfaces may comprise a digital input output interface, an analog input output interface, a CANopen interface, a USB (5V,GND, Sig1,2) interface, and a programmable MCU interface. The robotic medical surgical system may further comprise sensors or sound devices such as mic/speakers for voice recognition connected to the MCU or CANopen interface the docking system, wherein the sensors are configured to be programmed and controlled through the MCU or CANopen interface. In another embodiment, the docking connector only requires CAN data communication and a main power source through the robotic surgical arm. Further, the electrical interfaces may be connected to the robotic surgical end effector interface and the docking connector may be programmed as input output for the robotic surgical arm.

The present invention provides a quick KUKA iiwa Med compatible docking system that allows motorized surgical end-effectors with sensing devices to be mounted.

In a preferred embodiment, the present invention is a docking system that allows motorized surgical end-effectors with sensing devices to be mounted to Kuka iiwa med series quickly without needing to configure separate connections to the motion controllers, sensor interfaces. The docking system includes motion controllers that can control brushed/brushless motors with encoders up to 3, digital IO (input output) up to 10, analog IO up to 10, emergency switches, enabling push buttons up to 2, CANopen interface, 1 USB (5V,GND, Sig1,2) interface and programmable MCU interface. CANopen is a standardized interface format for devices, interfaces, and applications, enabling device manufacturers to create CANopen devices that plug and play. An MCU is an intelligent semiconductor IC that consists of a processor unit, memory modules, communication interfaces and peripherals. Any sensors or sound devices such as mic/speakers for voice recognition can be connected to the docking system and can be programmed and controlled through the MCU/CANopen interface. The docking system only requires CAN data communication and a main power source through the KUKA's tool flange connections to the end-effector console. It also includes a mechanical quick mount for Viky systems (510 k #) and handles manipulating the arm with hand-guidance. The IOs can be connected to KUKA Sunrise interface so that the docking interface is programmed as IO for KUKA iiwa arm.

Still other aspects, features, and advantages of the present invention are readily apparent from the following detailed description, simply by illustrating a preferable embodiments and implementations. The present invention is also capable of other and different embodiments and its several details can be modified in various obvious respects, all without departing from the spirit and scope of the present invention. Accordingly, the drawings and descriptions are to be regarded as illustrative in nature and not as restrictive. Additional objects and advantages of the invention will be set forth in part in the description which follows and in part will be obvious from the description or may be learned by practice of the invention.

### BRIEF DESCRIPTION OF THE DRAWINGS

For a more complete understanding of the present invention and the advantages thereof, reference is now made to the following description and the accompanying drawings, in which:

FIG. 1 is a perspective view of a docking connector system of the present invention.

FIG. 2 is a top perspective assembly view of a docking connector system in accordance with a preferred embodiment of the present invention.

FIG. 3 is a bottom perspective assembly view of a docking connector system in accordance with a preferred embodiment of the present invention.

FIG. 4A is a top perspective view of a docking connector in accordance with a preferred embodiment of the present invention.

FIG. 4B is a front view of a docking connector in accordance with a preferred embodiment of the present invention.

FIG. 4C is a side view of a docking connector in accordance with a preferred embodiment of the present invention.

FIG. 46D is a top view of a docking connector in accordance with a preferred embodiment of the present invention.

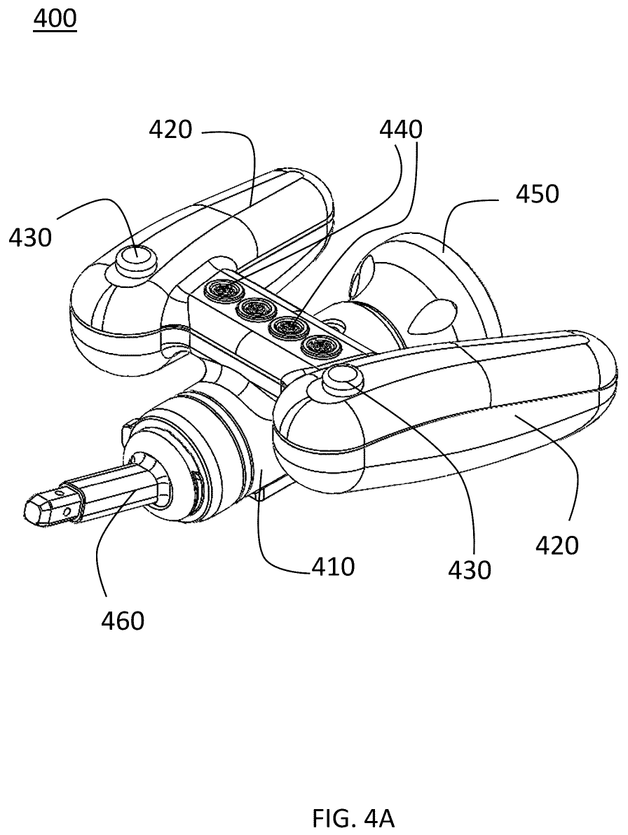

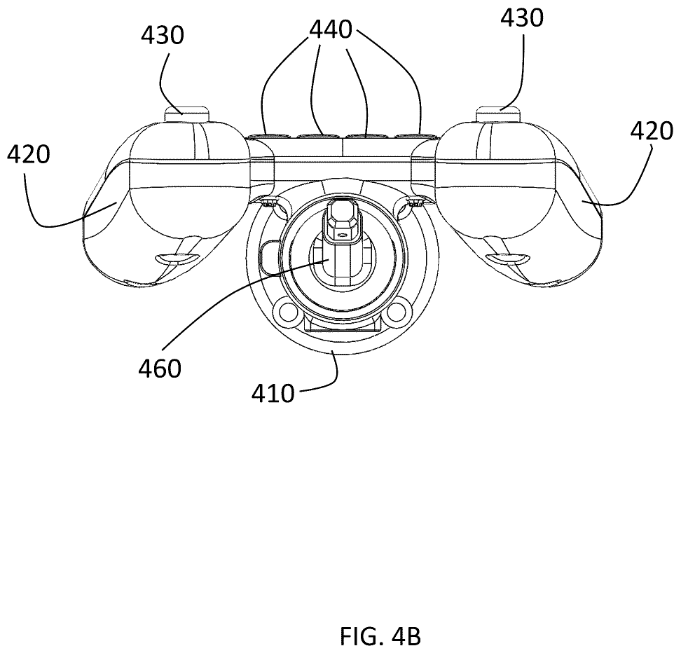

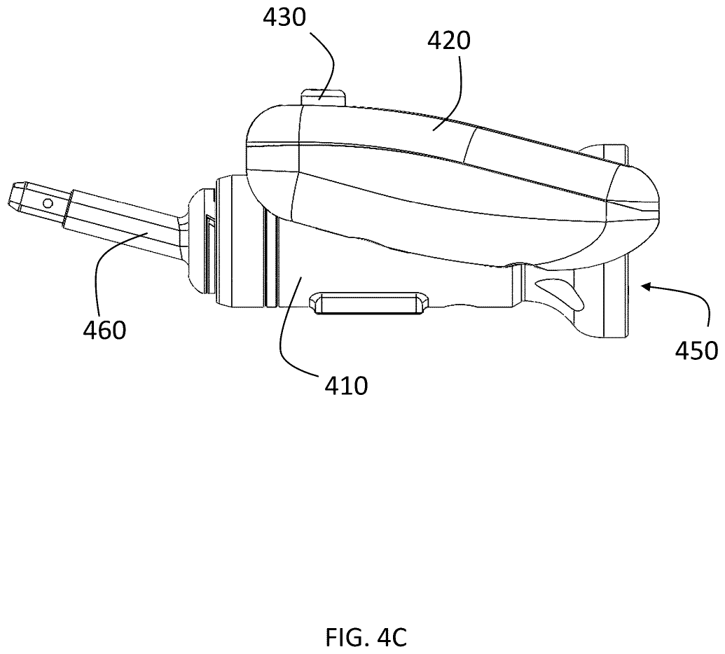

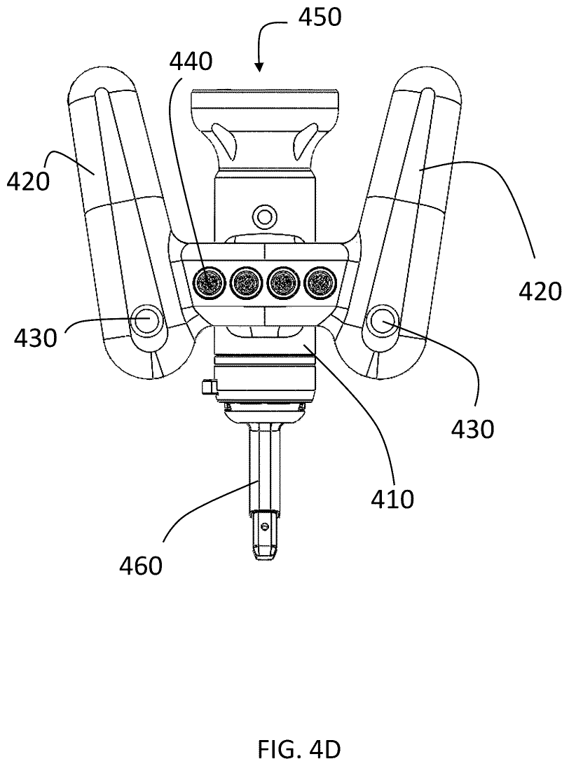

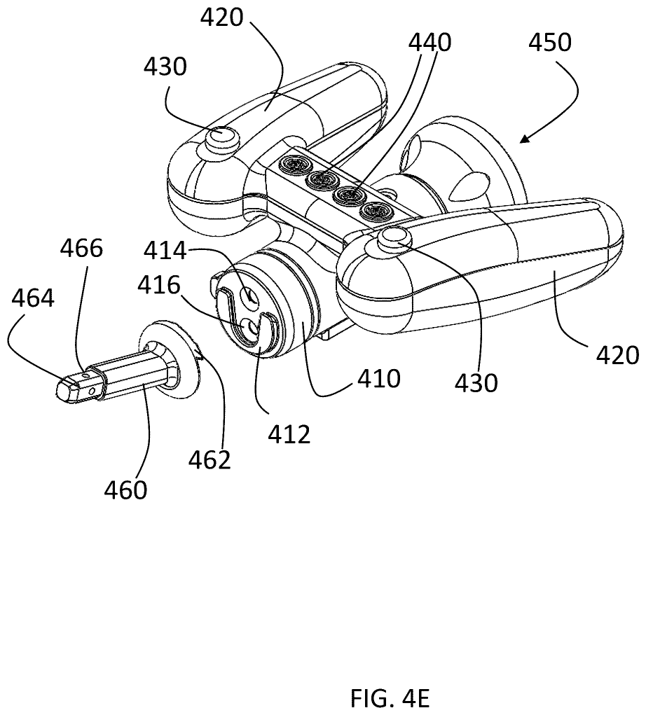

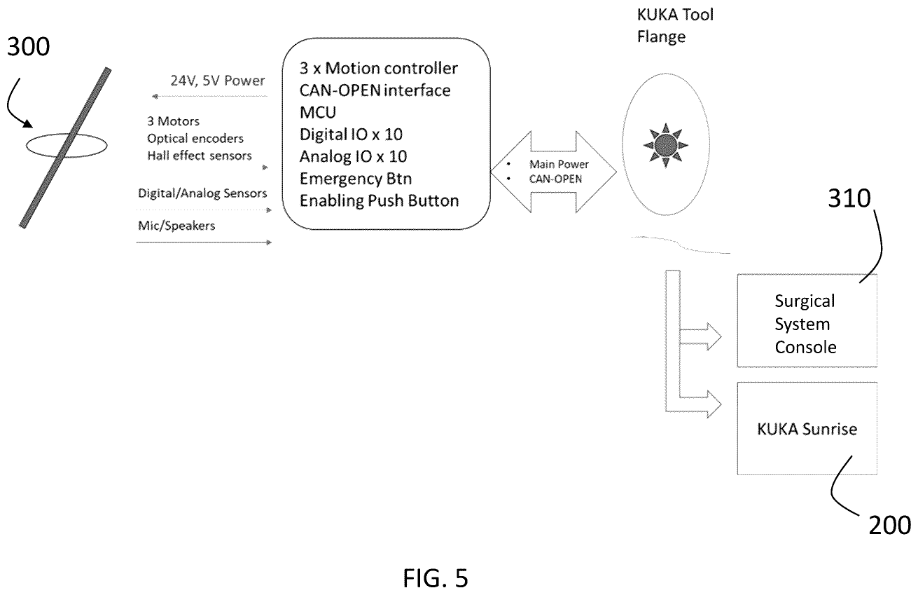

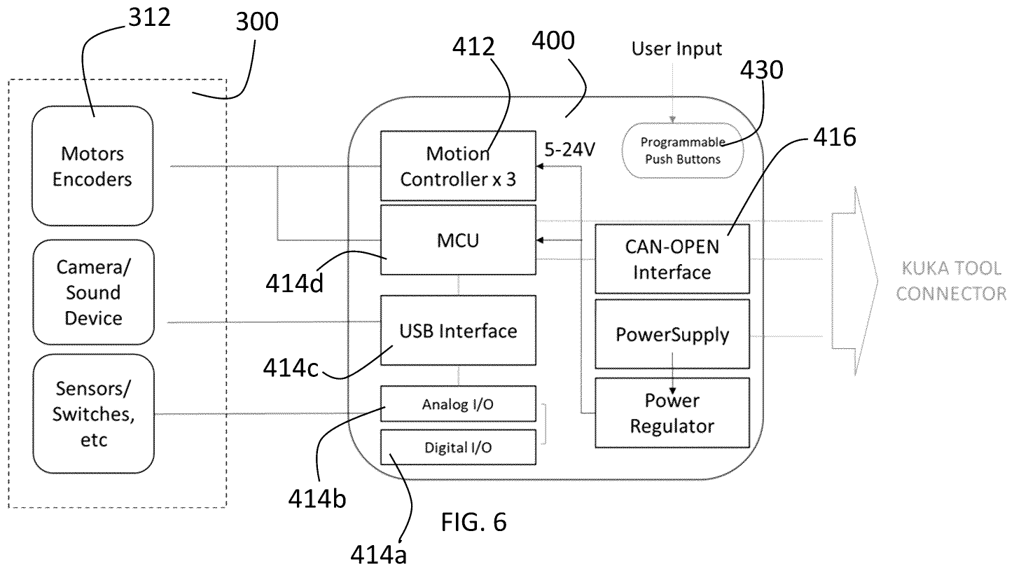

FIG. 4E is an assembly view of a docking connector in accordance with a preferred embodiment of the present invention.

FIG. 5 is a diagram summarizing a docking connector system of a preferred embodiment of the present invention.

FIG. 6 is a block diagram illustrating a system architecture of a docking connector system in accordance with the present invention.

### DETAILED DESCRIPTION OF THE PREFERRED EMBODIMENTS

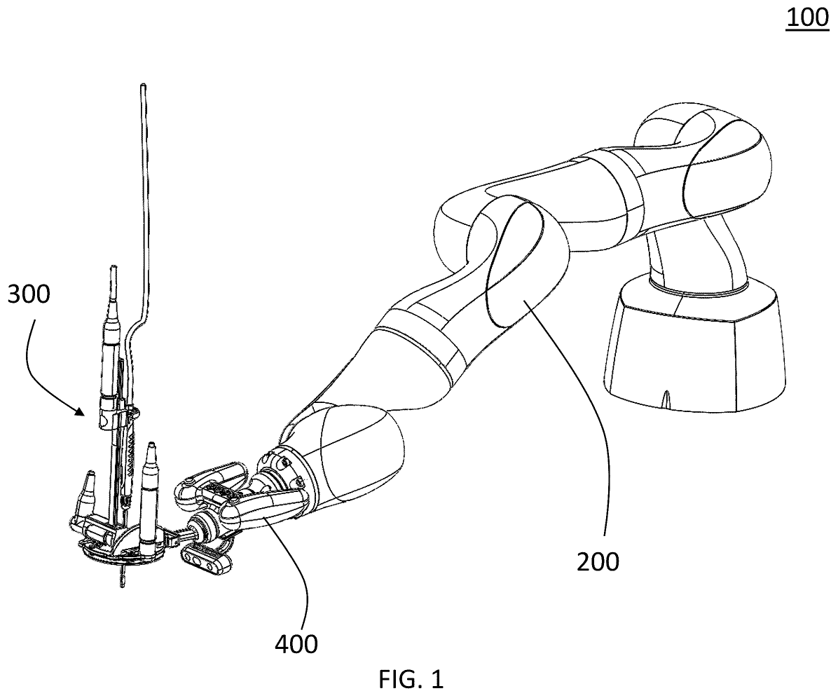

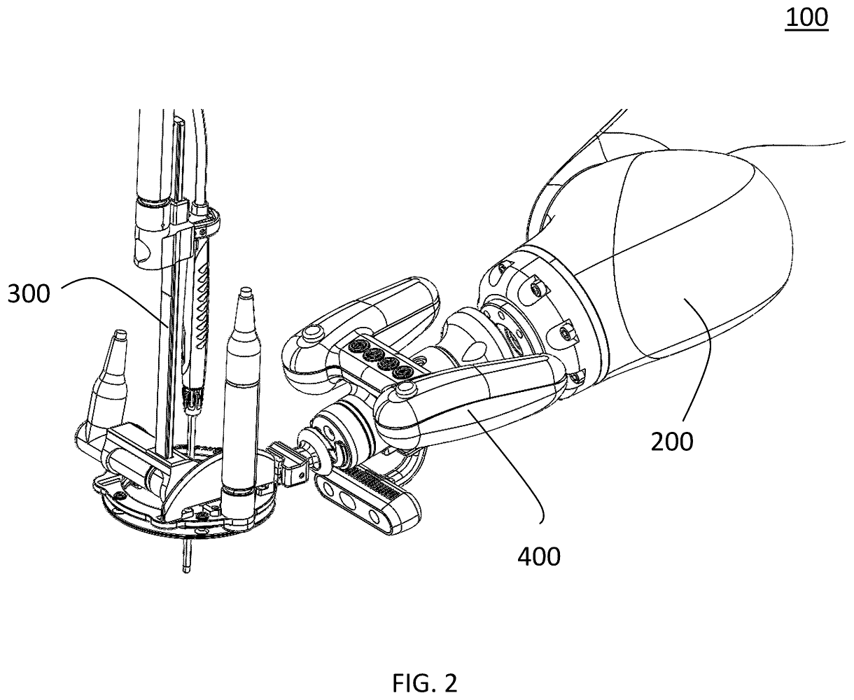

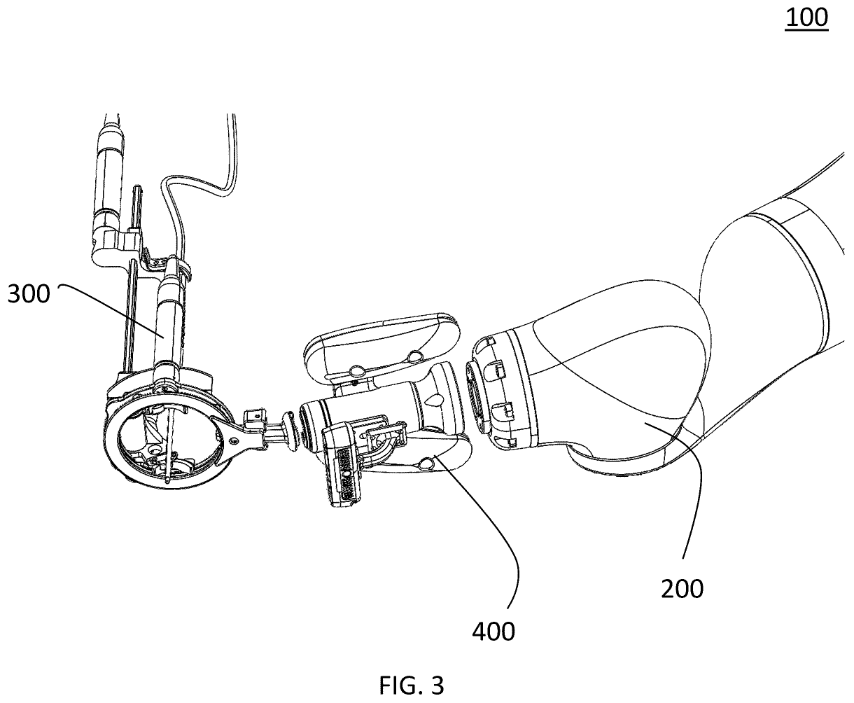

A preferred embodiment of the present invention is described with reference to the drawings. A preferred embodiment of a docking connector system of the present invention is shown in FIGS. 1-3. The system includes a robotic surgical arm 200 such as a KUKA iiwa arm, a robotic surgical end effector 300, and a docking connector 400 to connect the robotic surgical arm 200 to the surgical system 300. The robotic surgical end effector may be a system such as is described in U.S. Published Patent Application No. 2024/0156533 entitled “Robotic Cold Atmospheric Plasma Surgical System and Method.”

A docking connector 400 of a preferred embodiment is described with reference to FIGS. 4A-4E. The docking connector has a housing 410, which houses the printed circuit boards (PCB's) of the present invention. The housing has a pair of handles 620, which can be used to maneuver the robotic or mechanical arm 200 into position to performed a procedure. Each handle 620 has a programmable push button 630 for providing an operator with various controls associated with the system. Further, a plurality of cable connectors 640, four in the embodiment shown in FIGS. 6A-6E, are mounted in the housing to provide operational connections to the robotic surgical end effector 300.

On the rear of the docking connector 400 there is a conventional connector 450 to the robotic surgical arm 200. For example, is can be a mechanical kuka flange screw mount (8×ISO 9409-1-50-7-M6.) and/or electrical kuka media flange (data transfer/voltage/gnd).

On the front of the docking connection 400 there is a mechanical connector 460 to the robotic surgical end effector 300. The housing has a lip or flange 412 that mates with a corresponding lip or flange 442 on the mechanical connector 660. The housing has screw holes 414/416 that receive screws (not shown) when assembling the housing. The mechanical connector 660 may have a square shape with a chamfer at the tip to provide alignment with a female mount in the robotic surgical end effector 300 and a locking mechanism 466 so holding the docking connector 400 and robotic surgical end effector 300 together.

As shown in FIG. 5, the docking connector includes motion controllers 412 that can control brushed/brushless (2-3 phases) motors 312 with encoders up to 3, digital IO (input output) 414a (up to 10), analog IO 414b (up to 10), emergency switches, enabling push buttons up to 2, CANopen interface, 1 USB (5V,GND, Sig1,2) interface 414c and programmable MCU interface 414d. Any sensors or sound devices such as mic/speakers for voice recognition can be connected to the docking system and can be programmed and controlled through the MCU/CANopen interface 416. The motion controllers 412 can control motor voltage up to 30V, and also are capable of con motor position control. The IOs can be connected to KUKA Sunrise interface so that the docking connector is programmed as IO for KUKA iiwa arm.

The present invention has numerous advantages over prior systems, including, but not limited to, quick mount for robotic end-effectors, easy to deal with sterilization, no wire arrangement needed for Robotic end-effector with sensors and motors, and scalability thanks to digital IOs, CANopen, programming interface.

The foregoing description of the preferred embodiment of the invention has been presented for purposes of illustration and description. It is not intended to be exhaustive or to limit the invention to the precise form disclosed, and modifications and variations are possible considering the above teachings or may be acquired from practice of the invention. The embodiment was chosen and described to explain the principles of the invention and its practical application to enable one skilled in the art to utilize the invention in various embodiments as are suited to the particular use contemplated. It is intended that the scope of the invention be defined by the claims appended hereto and their equivalents. The entirety of each of the aforementioned documents is incorporated by reference herein.

What is claimed is:

1. A robotic medical surgical system docking connector comprising:
a housing comprising:
a first and second handles;
a programmable push button at least on of said first and second handles; and
a plurality of cable connectors;
a plurality of controllers in said housing;
a plurality of electrical interfaces in said housing
an interface for connecting said housing to a robotic surgical arm; and
a mechanical interface for connecting said housing to a robotic surgical end effector.

2. A robotic medical surgical system docking connector according to claim 1, wherein said plurality of electrical interfaces comprise:
a digital input output interface;
an analog input output interface;
a CANopen interface;
a USB (5V,GND, Sig1,2) interface; and
a programmable MCU interface.

3. A robotic medical surgical system comprising:
a robotic surgical arm;
a docking connector comprising:
a housing comprising:
a first and second handles;
a programmable push button at least on of said first and second handles; and
a plurality of cable connectors;
a plurality of controllers in said housing;
a plurality of electrical interfaces in said housing;
an interface connecting said housing to said robotic surgical arm; and
a mechanical interface for connecting said housing to a robotic surgical end effector;
a robotic surgical end effector mechanically connected to said mechanical interface in said docking connector; and
a plurality of electrical cables connecting said robotic surgical end effector to said electrical interfaces in said docking connector.

4. A robotic medical surgical system according to claim 3, wherein said plurality of electrical interfaces comprise:
a digital input output interface;
an analog input output interface;
a CANopen interface;
a USB (5V,GND, Sig1,2) interface; and
a programmable MCU interface.

5. A robotic medical surgical system according to claim 4, further comprising sensors or sound devices such as mic/speakers for voice recognition connected to the MCU or CANopen interface the docking system, wherein the sensors are configured to be programmed and controlled through the MCU or CANopen interface.

6. A robotic medical surgical system according to claim 5, wherein the sensors comprise sound devices.

7. A robotic medical surgical system according to claim 6, wherein the sound devices comprise a microphone and a speaker.

8. A robotic medical surgical system according to claim 4, wherein the docking connector only requires CAN data communication and a main power source through the robotic surgical arm.

9. A robotic medical surgical system according to claim 4, wherein the electrical interfaces are connected to the robotic surgical end effector interface and the docking connector is programmed as input output for the robotic surgical arm.
